(19) United States
(12) Patent Application Publication
(10) Pub. No.: US 2026/0357994 A1
(43) Pub. Date: Nov. 12, 2026

(54) COOKING ASSEMBLY FOR ELIMINATING VOLATILE COMPOUNDS THAT CAUSE THE EMISSION OF MALODORS

(71) Applicant: TANATAR KALIP PRES ISLERI SANAYI VE TICARET LIMITED SIRKETI, Eskisehir (TR)

(72) Inventors: Sedat HALAC, Eskisehir (TR); Fatma UNUGUR, Eskisehir (TR)

(73) Assignee: TANATAR KALIP PRES ISLERI SANAYI VE TICARET LIMITED SIRKETI, Eskisehir (TR)

(21) Appl. No.: 19/139,384

(22) PCT Filed: Jan. 23, 2023
(86) PCT No.: PCT/TR2023/050047

§ 371 Date: Jun. 16, 2025

(30) Foreign Application Priority Data
Dec. 30, 2022 (TR) 2022/021712

(51) Int. Cl. F24C 15/14; A47J 27/00; A61L 9/18; F24C 15/24

(52) U.S. Cl. CPC F24C 15/14; A47J 27/004; A61L 9/18; F24C 15/24

(57) ABSTRACT

The provided is particularly a cooking assembly that allows the elimination of volatile components produced during the cooking of food products such as meat, poultry, fish, etc., resulting in less odor emission, unlike other cooking devices in the state of art, and to a cooking device including this cooking assembly.

### TECHNICAL FIELD

The invention relates in particular to a cooking assembly that allows the elimination of volatile components produced during the cooking of food products such as meat, poultry, fish, etc., resulting in less odor emission, unlike other cooking devices in the state of art, and to a cooking device comprising this cooking assembly.

### BACKGROUND

As is in the state of the art, some volatile components are formed while some foods such as meat, chicken, and fish are cooked. One of the reasons for the odors that occur during cooking is the appearance of the said volatile components. These volatile components are usually compounds such as aldehyde, carboxylic acid, ester, furan furanone, imidazole, pyrazine, pyridine, pyrrole, and thiazole.

Removing these volatile components that cause odor during cooking from the cooking environment is especially important for making the dining areas more comfortable. While it is much easier to get rid of these odors in home kitchens due to the cooking of smaller-scale meals, in very large-scale food cooking areas such as restaurants, production halls, or sales areas, it negatively affects the comfort of people in the environment. In order to get rid of the odors well described in the art, ventilation systems are generally built on a very large scale and at high costs. It is also a predictable technical problem that the maintenance costs of these ventilation systems will be high.

In order to eliminate the above-mentioned technical problems, it has become imperative to innovate in the relevant technical field and to carry out research and development activities by those skilled in the art.

### BRIEF DESCRIPTION OF THE INVENTION

In the art, although there are no limitations with these, odors that arise during the cooking of foods such as meat, chicken, and fish should be removed to ensure the comfort of ambient life. It is the volatile components that cause the said odors. Removing these volatile components known in the art from the environment often has a large footprint and high-cost ventilation systems. Due to the fact that these ventilation systems create sales and maintenance costs and also take up a lot of space, the present inventors offer technical solutions and advantages for the relevant technical field. Accordingly, the present invention relates in particular to a cooking assembly that allows the elimination of volatile components produced during the cooking of food products such as meat, poultry, fish, etc., resulting in less odor emission, unlike other cooking devices in the state of the art, and to a cooking device comprising this cooking assembly.

### BRIEF DESCRIPTION OF THE FIGURES

FIG. 1 shows a representative view of a cooking device comprising the cooking assembly of the invention.

### BRIEF DESCRIPTION OF REFERENCES

1 Cooking Device
10 Upper Body
11 Infrared Filament
20 Lower Body
21 Cooking Chamber
30 Food Product

### DETAILED DESCRIPTION OF THE INVENTION

In this detailed description, the subject matter of the invention relates in particular to a cooking assembly, which enables the elimination of volatile components, especially during the cooking of food products (30) such as meat, poultry, fish, etc., resulting in the emission of less odor, unlike other cooking devices in the present art, and to a cooking device (1) comprising such a cooking assembly, and is described only by means of examples that will not have any limiting effect for a better understanding of the subject matter.

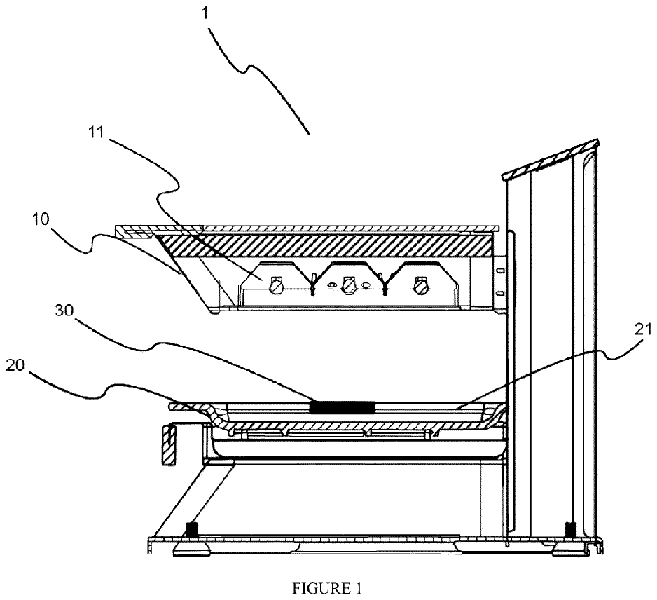

FIG. 1 shows the cooking device of the present invention. The cooking device (1) of the invention consists of two structures, mainly the lower body (20) and the upper body (10).

The upper body (10) within the cooking device (1) contains elements for the provision of cooking food products. In the present invention, cooking operations are provided with at least one infrared filament(s) (11) positioned in the upper body (10). The scope of protection of the present invention is not narrowed by the structure, shape, and number of the infrared filament(s) (11).

The upper body (10) has a feature that moves up and down in a vertical position towards the lower body (20) (in the y-axis to the coordinate system). This technical feature of the upper body (10) makes it possible to obtain the desired temperature for the food product (30) to be cooked. By bringing the high temperature-producing infrared filaments (11) closer to the food product (30) to be cooked, it will be possible to achieve higher temperatures as can be predicted. Furthermore, this temperature obtained may allow cooking operations to be carried out in shorter periods of time for the food product (30) intended to be cooked. Here, the decision about at what level or distance the upper body (10) can be approximated towards the lower body (20) varies according to the product to be cooked and the temperature and time desired to be cooked. Also herein, the upper body (10) is composed of up or down adjustable steps, and a preferred step may remain constant at the determined times and temperatures. The height-adjustable steps of the upper body (10) herein are controllable by a simple control unit, which may have a fully automated system.

The cooking device (1) of the invention comprises at least one lower body (20) comprising at least one cooking chamber (21) prepared for the food to be cooked and at least one oil-water chamber required for the cooking medium. If preferred, the number of oil-water reservoirs may be more than one. The oil-water reservoir is positioned next to the lower part of the lower body (20). In the invention, the oil-water reservoir allows the cooked food product (30) to go to the water-oil reservoir under the cooking grate of the juice and to prevent the burning of fat, thus preventing odor outflow from the food products (30).

If preferred in the invention, the cooking device (1) may include more than one upper body (10) and lower body (20) that allow many cooking processes to take place at the same time and temperature. There may even be more than one upper body (10) and lower body (20), and there may be a thermal barrier between the multiple upper bodies (10) and lower bodies (20). In this way, different food products (30) can be cooked separately in the said assemblies.

In a possible embodiment of the invention, the lower body (20) is also movable in a stepwise up-and-down manner.

As is in the state of the art, especially during the cooking of food products (30) such as meat, chicken, and fish, there are bad odors caused by food products (30), oil, and/or water used. The cause of the mentioned bad odors is the presence of volatile components that appear during cooking. These volatile components are usually compounds such as aldehyde, carboxylic acid, ester, furan furanone, imidazole, pyrazine, pyridine, pyrrole, and thiazole. These odor-emitting compounds will be removed from their odor-emitting functional structures if they reach decay temperatures. The inventors of the present invention have determined that it is possible to remove the volatile components that emit bad odors from these structures by increasing them to their deterioration temperatures. In Table 1, the degradation temperatures of the said volatile compounds are shared.

TABLE 1
Decomposition temperatures of volatile components
that emit a foul odor during cooking.

| Volatile Components | Distortion Temperature | |
|---|---|---|
| Aldehyde | 480-525 | °C |
| Carboxylic acid | 223-373 | °C |
| Ester | 900-1300 | °C |
| Furan | 750-1000 | °C |
| Furanone | 600 | °C |
| Imidazole | 340-500 | °C |
| Pyrazine | 140-385 | °C |
| Pyridine | 900-1050 | °C |
| Pyrrole | 550 | °C |
| Thiazole | 190 | °C |

The cooking device (1) of the present invention comprises at least one infrared filament (21) which, as mentioned earlier, is preferably capable of moving in an upward or downward direction in a stepwise manner and which performs the cooking operations having at least one in its structure. These infrared filaments (11) provide a cooking temperature of up to 900 °C. Of course, the position of the upper body (10) can be adjusted according to the food product (30) and the time to be cooked, and thus different cooking temperatures can be provided for the product (30) to be cooked. During cooking from the product to be cooked placed in the lower body (20), the volatile components move towards the upper body (10). Preferably, the cooking device (1) may comprise apparatus for the upward movement of these volatile components.

Although the temperature varies according to the food product (30) to be cooked in the lower body (20), the temperature values can reach up to 900 °C in the regions close to the upper body (10) according to the position of the upper body (10). Accordingly, there is a gradual temperature increase from the lower body (20) to the upper body (10). Accordingly, deterioration of their functional structure will be carried out at distortion temperatures as the volatile components move from the lower body (20) towards the upper body (10). The foul-smelling properties of the volatile components, whose functional structures are impaired, will also be eliminated. In particular, the infrared filament (11) exhibits superior performance at temperatures up to 900 °C. In this way, if the deterioration temperatures of the volatile components up to this temperature are in question, the bad odors of the volatile components reaching the deterioration temperatures will be eliminated.

The cooking device (1) of the invention works as follows; the food product (30) to be cooked is added to the cooking chamber (21) within the lower body (20). The upper body (10) is fixed gradually by moving it up and down for the targeted temperature and time for the food product (30). The processes for cooking are initiated, and the volatile components from the lower body (20), which emit a foul odor, move toward the upper body (10). The infrared filament (11) in the upper body (10) is capable of generating temperatures of up to 900 °C; this temperature gradually decreases up to the lower body (20). The volatile components are exposed to higher temperatures as they move from the lower body (20) toward the upper body (10). The volatile components that reach the deterioration temperature will be removed from their functional structures that cause bad odors by deterioration and in this way, the spread of bad odors from the cooking device (1) will be prevented.

The protection scope of the invention is specified in the appended claims and cannot be strictly limited to what is described in this detailed description for illustrative purposes. It is clear that those skilled in the art may exhibit similar embodiments in light of the foregoing without departing from the main theme of the invention.

What is claimed is:

1. A cooking device having an assembly comprising:
at least one upper body comprising at least one infrared filament, wherein the infrared filament provides a temperature required for cooking food products within the upper body and the upper body is moveable up and down in a vertical position, wherein the vertical position is in y-axis of a coordinate system, wherein the ensured is that a targeted temperature and cooking time for the food product to be cooked are gradually adjustable;
at least one lower body containing at least one cooking chamber for placement of the food product to be cooked within
and allowing functional structures to deteriorate at the temperatures provided to the cooking device by the infrared filaments in the upper body during movement of bad odor emitting volatile components occurring during cooking of the food product in the lower body from the lower body to the upper body.

2. The cooking device having the assembly according to claim 1, wherein the infrared filaments provide a temperature of up to 900 °C for degradation of the functional structures of the bad odor emitting volatile components having a degradation temperature of up to these temperatures.

3. The cooking device having the assembly according to claim 1, comprising more than one upper body.

4. The cooking device having the assembly according to claim 1, comprising more than one lower body.

5. The cooking device having the assembly according to claim 1, wherein the lower body is moveable in a stepwise up and down manner.

6. The cooking device having the assembly according to claim 1, comprising at least one oil-water reservoir positioned in lower vicinity of the lower body, collecting juices of the food product being cooked and preventing burning of fat, wherein emission of odors from the food product is prevented.
